(19) United States
(12) Patent Application Publication
(10) Pub. No.: US 2005/0268920 A1
(43) Pub. Date: Dec. 8, 2005

(54) Medical information carrying and presenting system

(76) Inventor: Vivek Sharma, Melbourne, FL (US)

Correspondence Address: Mr. Vivek Sharma, 4796-1 Lake Waterford Way West, Melbourne, FL, 32901, US

(21) Appl. No.: 11/047,506

(22) Filed: Feb. 1, 2005

Related U.S. Application Data
(60) Provisional application No. 60/577,456, filed on Jun. 7, 2004.

(51) Int. Cl. A61B 5/00; A61B 19/00

(52) U.S. Cl. 128/897; 128/898

(57) ABSTRACT

A universal system and method to permanently carry and make available critical personal medical information. Specifically, common Allergic drug reactions and permanent chronic body illnesses or conditions. Useful to medical personnel, in emergency situations when the patient can't speak for himself.

### CROSS-REFERENCE TO RELATED APPLICATIONS

This application claims the benefit of provisional patent application Ser. No. 60/577,456, filed 2004 Jun. 7 by the present inventor.

### FEDERALLY SPONSORED RESEARCH

Not Applicable

### SEQUENCE LISTING OR PROGRAM

Not Applicable

### BACKGROUND OF THE INVENTION—FIELD OF INVENTION

Personal medical information carrying systems.

### BACKGROUND OF THE INVENTION—REFERENCES

U.S. Pat. Nos. 2,138,596; 3,790,775; 4,179,833; 4,256,459; 4,296,459.

### BACKGROUND OF THE INVENTION—PRIOR ART

Wallet Cards and Arm Bracelets/Pendants with medical information on/in them.

Disadvantages: People forget to wear/carry them.
In emergencies they can easily get separated from the patient.
Electronic Pendants require auxiliary hardware to retrieve info.
Information retrieval time is too slow for an emergency situation.

### SUMMARY

A Way to make ones critical medical information available in emergency situations.

### DRAWINGS

FIG. 1 shows symbols marked behind a person's Ear.

### DETAILED DESCRIPTION

A universal system to permanently carry and present critical medical information about oneself. Critical medical information specifically includes, common Allergic drug reactions and permanent chronic body illnesses or conditions. This will help prevent patients from receiving unsuitable treatment. This information is needed by medical personnel, when the patient might be unconscious or incapacitated.

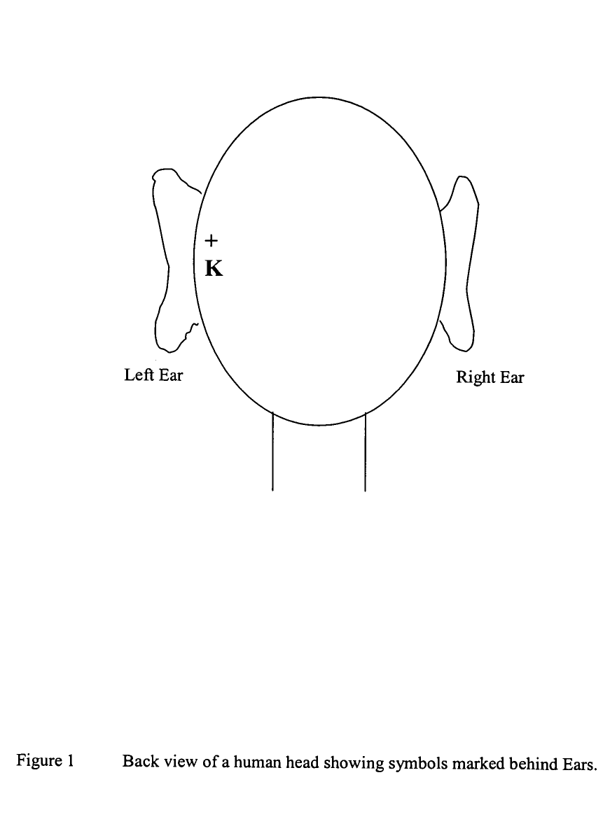

A set of symbols representing major medical conditions will be tattooed on the patient's body, behind the Ears. See FIG. 1. These standardized symbols are not language specific. The symbols will be located behind the Ears, so that the information will generally be hidden, but Medical and emergency personnel can look at it easily by folding back the Ear. Also this location will protect the tattoo from sun damage and deterioration over time. Allergies will be marked behind Left Ear, and Medical conditions behind Right Ear. Most patients will need only a couple of symbols, so there is enough space behind the Ears. Patients can also choose inner upper arm location, but its not recommended.

Contemporary Black fine line stencil tattooing technique will be used to imprint the symbols. A licensed practitioner can apply tattoos in a Hospital or offsite.

Standard current Laser removal techniques can be used if a medical condition changes, and a symbol has to be removed.

Symbols: A set of universally identifiable, easy to mark symbols for common conditions are listed below. Symbols are simple yet different enough so that one cannot be confused for the other, even if the tattoo is old, faded, or shriveled. Symbols will usually be in the sequence below (Top to bottom) to further reduce chance of error while reading. Symbol Size is 4×4 mm, with 2 mm space in between. There is room for addition of more symbols in the future, keeping within the framework of the current system.

| Behind Left Ear (Allergies) | Behind Right Ear (Conditions) |
|---|---|
| +-Penicillin | A-Asthma |
| Δ-ASA | E-Epilepsy |
| Z-Sulfonamides | H-HIV |
| S-Cephalosporins | P-Pacemaker |
| O-Opiates | T-Diabetes |
| K-Anesthetics | |

### DETAILED DESCRIPTION CONT.

This system has the following advantages over current art:
(a) Permanently carries information.
(b) Non-language specific, so usable worldwide.
(c) Ear location provides discrete yet quick access.
(d) No extra equipment or devices required to retrieve information.
(e) Information always stays with patient.

### CONCLUSION SCOPE AND RAMIFICATIONS

The system can also other parts of body for symbol location. Besides tattooing, other contemporary marking methods can be used. Standardization, overseen by a medical organization can lead to global use. Thus scope of the invention should be determined by the appended claims and their legal equivalents, rather than by the examples given.

1. I claim a method and system for carrying and instantly making available critical medical information about a patient, in emergency situations, comprising:
(a) Standardized symbols denoting medical conditions marked on a human body in an inconspicuous but easily accessible location.
(b) Method of permanently marking and removing the information.
(c) A system not requiring any auxiliary equipment to retrieve the information.
(d) A system that is not Language specific.
